(19) United States
(12) Patent Application Publication
(10) Pub. No.: US 2026/0232357 A1
(43) Pub. Date: Aug. 13, 2026

(54) SCREW FOR FEEDING, SPACING AND/OR ORIENTATION OF CONTAINERS IN A PACKAGING INSTALLATION

(71) Applicant: SIDEL PARTICIPATIONS, Octeville-sur-Mer (FR)

(72) Inventors: Théo BURDY, OCTEVILLE- SUR-MER (FR); Julien DURIGON, OCTEVILLE- SUR-MER (FR)

(21) Appl. No.: 19/539,828

(22) Filed: Feb. 13, 2026

(30) Foreign Application Priority Data
Feb. 13, 2025 (FR) 2501503

(51) Int. Cl. A61B 17/70; A61B 17/86

(52) U.S. Cl. CPC A61B 17/7032; A61B 17/8625; A61B 17/863; A61B 17/864

(57) ABSTRACT

Described is a unit for a screw for feeding, setting the pitch of and/or setting the orientation of containers. The unit includes a central body extending from an upstream end to a downstream end along a longitudinal axis, and a substantially helical thread rigidly attached to the body, the thread comprising a plurality of helixes, each helix having a crest, an upstream flank and a downstream flank, the thread being configured to be engaged between the containers. The unit includes for each helix at least a first substantially helical groove at the level of the upstream flank of the helix to receive a first wear ring, and a second substantially helical groove at the level of the downstream flank of the helix to receive a second wear ring. Also described is a feed screw, an installation for processing containers, and a method of feeding an entry of a processing machine.

### CROSS-REFERENCE TO RELATED APPLICATIONS

This application claims the benefit of French Application No. FR2501503, filed February 13. 2025, the entire contents of which is hereby incorporated herein by reference.

### BACKGROUND

In the field of processing containers, in particular in installations for the manufacture and packaging of drinks, it is necessary to feed each processing station with containers in an orderly manner and at a predefined rate. The containers must be fed to a processing machine via a container entry or a container feed such that the distance between two successive containers corresponds to the throughput—or processing speed—of the machine. Also, a modification of the spacing interval of the successive containers during passage from one processing station to a following processing station may be required. Furthermore, it may be necessary to modify the orientation of the containers before entry into a processing machine, for example before entry into a filling machine or a labelling machine.

For orderly feeding and for spacing or separating the containers at a particular pitch (e.g. at the distance corresponding to the throughput of the machine), it is known to use a feed screw system adapted to pivot on itself, thus making it possible to increase the pitch between two containers fed successively. It is also known to use such a screw for the separation of the containers, disposed alongside a transport path for the containers and therefore adapted to be driven in rotation. One of the problems with this type of feed screw is that it is liable to leave scratch marks on the body of the container transported in this way. Accordingly, it has been found that needs exist for an installation that does not degrade the appearance nor the integrity of the containers. It is to the provision of meeting these and other needs that the present disclosure is primarily directed.

### SUMMARY

Embodiments of the present disclosure provide for feeding screws, screw units for feeding screws, conveyor installations including feeding screws, and methods for feeding containers utilizing feeding screws.

An embodiment of the present disclosure includes a unit for a screw for feeding, setting a pitch of and/or setting an orientation of containers. The unit can include a central body extending from an upstream end to a downstream end along a longitudinal axis. The body includes a substantially helical thread rigidly attached thereto, the thread comprising a plurality of helixes, each helix having a crest, an upstream flank and a downstream flank, the thread being configured to be engaged between the containers. Each helix includes at least two grooves: a first substantially helical groove at a level of the upstream flank of the helix configured to receive a first wear ring, and a second substantially helical groove at a level of the downstream flank of the helix configured to receive a second wear ring.

An embodiment of the present disclosure also includes a screw comprising a central shaft inserted into the body of a unit as provided above.

An embodiment of the present disclosure also includes a conveyor installation for processing containers. The conveyor installation can include a conveyor device for conveying the containers, an entry of a processing machine, and at least one rotary shaft. The installation also includes at least one screw as provided above, the screw being horizontal and extending from upstream to downstream in a direction along one side of the conveyor device, the at least one screw being fixed to the at least one rotary shaft in order to enable rotation.

These and other aspects, objects, features, and embodiments will become apparent to a person of ordinary skill in the art upon consideration of the following detailed description of illustrative embodiments exemplifying the best mode as presently perceived.

### BRIEF DESCRIPTION OF THE DRAWINGS

For a more complete understanding of the embodiments and the advantages thereof, reference is now made to the following description, in conjunction with the accompanying figures briefly described as follows:

FIG. 1 is a diagram of a view from above of one possible embodiment of a packaging installation, the containers being bottles;

FIG. 2 is a diagram of a view from above of one possible embodiment of a packaging installation, the containers being small jerrycans of substantially rectangular shape;

FIG. 3 illustrates a perspective view of one possible embodiment of a feed screw comprising a plurality of units;

FIG. 4 illustrates one possible embodiment of a feed screw comprising a plurality of helixes of constant pitch and diameter;

FIG. 5 illustrates one possible embodiment of a feed screw comprising a plurality of helixes of decreasing pitch and diameter;

FIG. 6 illustrates one possible embodiment of a feed screw unit including two grooves;

FIG. 7 illustrates another possible embodiment of a feed screw unit including two grooves;

FIG. 8 illustrates one possible embodiment of a feed screw unit including three grooves;

FIG. 9 illustrates a side view of one possible embodiment of a feed screw unit including three grooves; and

FIG. 10 illustrates a view in section of one possible embodiment of a feed screw unit.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

The following examples are put forth so as to provide those of ordinary skill in the art with a complete disclosure and description of how to perform the methods and use the devices and methods disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

The features, structures, or characteristics described above may be combined in one or more embodiments in any suitable manner, and the features discussed in the various embodiments may be interchangeable, if possible. In the following description, numerous specific details are provided in order to fully understand the embodiments of the present disclosure. However, a person skilled in the art will appreciate that the technical solution of the present disclosure may be practiced without one or more of the specific details, or other methods, components, materials, and the like may be employed. In other instances, well-known structures, materials, or operations are not shown or described in detail to avoid obscuring aspects of the present disclosure.

The terms used herein are intended to have their ordinary meaning unless specifically defined otherwise. Directional terms such as “upper,” “lower,” “front,” “back,” and similar terms are used for convenience and are not intended to be limiting unless the context clearly indicates otherwise. The use of “may,” “can,” “could,” and similar terms indicates possible embodiments and is not intended to limit the scope of the disclosure.

Although the relative terms such as “on,” “below,” “upper,” and “lower” are used in the specification to describe the relative relationship of one component to another component, these terms are used in this specification for convenience only, for example, as a direction in an example shown in the drawings. It should be understood that if the device is turned upside down, the “upper” component described above will become a “lower” component. When a structure is “on” another structure, it is possible that the structure is integrally formed on another structure, or that the structure is “directly” disposed on another structure, or that the structure is “indirectly” disposed on the other structure through other structures.

In this specification, the terms such as “a,” “an,” “the,” and “said” are used to indicate the presence of one or more elements and components. The terms “comprise,” “include,” “have,” “contain,” and their variants are used to be open ended, and are meant to include additional elements, components, etc., in addition to the listed elements, components, etc. unless otherwise specified in the appended claims.

The terms “first,” “second,” etc. are used only as labels, rather than a limitation for a number of the objects. It is understood that if multiple components are shown, the components may be referred to as a “first” component, a “second” component, and so forth, to the extent applicable.

In the field of processing containers, in particular in installations for the manufacture and packaging of drinks, it is necessary to feed each processing station with containers in an orderly manner and at a predefined rate.

In the context of the disclosure the containers are by way of non-limiting example bottles, cans, small bottles, jerrycans or cartons. Such a container is intended to contain a fluid, a liquid, powders or granules, notably of agriculture-foodstuffs or cosmetic type. This list is not exhaustive.

These containers can be made of any type of material, metal, glass, but preferably a plastic-based material, notably polyethylene (PE) or polypropylene (PP), conferring a flexibility rendering said containers semi-rigid.

Such containers can be any shape, symmetrical or otherwise, regular or irregular. Furthermore each of the containers can have a rounded section, of circular or oval overall shape, or a polygonal, notably rectangular or square section.

In particular, the use of containers of specific shape, for example asymmetrical or oblong, is widespread in the field of food, home care and personal care (FHPC) containers, for example for a container intended to contain a shower gel or shampoo, or in the field of home care and cleaning, such as a spray bottle for detergents and disinfectants.

In known manner said containers are moved along said production line, either one by one or grouped into batches, to be fed to various successive different processing stations, such as manufacture of the container, for example in an injection molding or stretching-blowing operation in the case of a plastic material bottle, followed by filling and then by closing by a stopper and labelling. At the end of these processes the containers are referred to as “finished”.

For handling them such finished containers are packaged in batches.

Each batch comprises a group of containers assembled in a row or in a matrix arrangement, generally of parallelepipedal overall shape, often square or rectangular, in a number of rows and columns. For example a standard batch groups six containers in two rows and three columns, often referred to as a pack.

Once the groups of containers have been produced the groups generally undergo packaging by boxing or wrapping, preferably in a film-wrapping step or in particular by wrapping with paper or card, in order to hold together the containers of the same batch.

During these various steps the containers are therefore transported along the production line in a movement direction extending longitudinally from upstream to downstream between and in the various stations dedicated to each process that the containers have to undergo.

In particular, the containers must be fed to a processing machine via a container entry or a container feed in such a manner that the distance between two successive containers corresponds to the throughput of the machine, that is to say its processing speed. Also there may be required a modification of the spacing interval of the successive containers during passage from one processing station to a following processing station.

Furthermore it may be necessary to modify the orientation of the containers before entry into a processing machine, for example before entry into a filling machine or a labelling machine.

For orderly feeding and for spacing or separating the containers at a particular pitch, that is to say at the distance corresponding to the throughput of the machine, it is known to have recourse to a feed screw system adapted to pivot on itself and for example making it possible to increase the pitch between two containers fed successively.

It is also known to use such a screw for the separation of the containers, disposed alongside a transport path for the containers and therefore adapted to be driven in rotation.

One of the problems with this type of feed screw is that it is liable to leave scratch marks on the body of the container transported in this way.

In effect a feed screw includes a thread, the crest of the thread coming into contact with the container to be fed, spaced and/or oriented, this contact then causing rubbing.

The disclosure advantageously proposes a feed screw unit and a feed screw in which the helical thread comprises at least two grooves, each of the at least two grooves being configured to receive a wear ring. The presence of the wear ring makes it possible to prevent direct contact between the container and the thread or with the body of the screw. The container being in contact with the wear ring, rubbing is reduced and the appearance or integrity of said container is not degraded.

The disclosure is directed first to a unit for a screw for feeding, setting the pitch of and/or setting the orientation of containers, said unit including:
a central body extending from an upstream end to a downstream end along a longitudinal axis,
a substantially helical thread rigidly attached to said body, said thread comprising a plurality of helixes, each helix having a crest, an upstream flank and a downstream flank, said thread being configured to be engaged between said containers.

The unit according to the disclosure is characterized in that it includes for each helix at least:
a first substantially helical groove at the level of the upstream flank of said helix adapted to receive a first wear ring, and
a second substantially helical groove at the level of the downstream flank of said helix adapted to receive a second wear ring.

Some embodiments of the feed screw unit include a third substantially helical groove at the level of the downstream flank of said helix adapted to receive a third wear ring.

In some embodiments the screw unit is produced in one piece.

In possible embodiments the screw unit is produced by 3D printing and is preferably made of polyamide.

In some embodiments the at least two grooves have the same helical profile as the helix.

In accordance with a possible additional feature in an axial plane perpendicular to the longitudinal axis of the body of the screw unit, the upstream angle of the upstream flank of the helix is identical to the downstream angle of the downstream flank of said helix.

The disclosure is also directed to a screw for feeding, setting the pitch of and/or setting the orientation of containers, said screw extending from an upstream end to a downstream end. The screw according to the disclosure is characterized in that it includes:
a central shaft,
at least one unit as described above, the shaft being inserted in the body of said unit.

In preferred embodiments the screw comprises at least two units, said units being abutted against one another by means of said central shaft inserted in the body of each of said at least two units so that the at least two grooves of each of said at least two units extend continuously from the upstream end to the downstream end.

In accordance with a possible additional feature the at least two grooves include one fixing point for a wear ring.

In some embodiments the screw includes at least two wear rings, said at least two wear rings having a shape complementary to the shape of the respective groove and extending the whole length thereof.

In accordance with a possible additional feature the at least two wear rings are made of polymer, preferably of polytetrafluoroethylene.

In some embodiments from the upstream end to the downstream end the helixes forming the thread have a decreasing radius and the pitch separating two successive helixes decreases.

The disclosure is further directed to an installation for processing containers including at least:
a device for conveying said containers,
an entry of a processing machine,
at least one rotary shaft.

The installation according to the disclosure is characterized in that it includes at least one feeding, pitch setting and/or orientation setting screw as described above, said screw being horizontal and extending from upstream to downstream in a direction along one side of said conveyor device, said at least one screw being fixed to said at least one rotary shaft in order to enable it to rotate.

In accordance with a possible additional feature the installation further includes a guide rail on the opposite side of and along said conveyor device, said rail facing said screw.

Another embodiment of the installation includes a second rotary shaft and a second screw, said second screw being on the opposite side of the conveyor device and extending in the upstream to downstream direction, said second screw being fixed to said second rotary shaft connected to said second screw in order to enable it to rotate, the two screws being symmetrical and configured to turn in opposite directions.

The disclosure also includes a method for feeding with containers an entry of a machine for processing said containers, said method including at least the following steps:
transporting the containers on a conveyor device,
setting the pitch of and/or setting the orientation of said containers before their entry into the processing machine, each body of a container being adapted to engage at the level of the upstream end a screw between two helixes formed on the perimeter of the body of said screw.

The method according to the disclosure is characterized in that the step of setting the pitch and/or setting the orientation of the containers is implemented by means of at least one screw as described above.

### General Discussion

In the field of processing containers, in particular in installations for the manufacture and packaging of drinks, it is necessary to feed each processing station with containers in an orderly manner and at a predefined rate.

In the context of the disclosure the containers are by way of non-limiting example bottles, cans, small bottles, jerrycans or cartons. Such a container is intended to contain a fluid, a liquid, powders or granules, notably of agriculture-foodstuffs or cosmetic type. This list is not exhaustive.

These containers can be made of any type of material, metal, glass, but preferably a plastic-based material, notably polyethylene (PE) or polypropylene (PP), conferring a flexibility rendering said containers semi-rigid.

Such containers can be any shape, symmetrical or otherwise, regular or irregular. Furthermore, each of the containers can have a rounded section, of circular or oval overall shape, or a polygonal, notably rectangular or square section.

In particular, the use of containers of specific shape, for example asymmetrical or oblong, is widespread in the field of food, home care and personal care (FHPC) containers, for example for a container intended to contain a shower gel or shampoo, or in the field of home care and cleaning, such as a spray bottle for detergents and disinfectants.

In known manner said containers are moved along said production line, either one by one or grouped into batches, to be fed to various successive different processing stations, such as manufacture of the container, for example in an injection molding or stretching-blowing operation in the case of a plastic material bottle, followed by filling and then by closing by a stopper and labelling. At the end of these processes the containers are referred to as “finished”.

For handling them such finished containers are packaged in batches.

Each batch comprises a group of containers assembled in a row or in a matrix arrangement, generally of parallelepipedal overall shape, often square or rectangular, in a number of rows and columns. For example, a standard batch groups six containers in two rows and three columns, often referred to as a pack.

Once the groups of containers have been produced the groups generally undergo packaging by boxing or wrapping, preferably in a film-wrapping step or in particular by wrapping with paper or card, in order to hold together the containers of the same batch.

During these various steps the containers are therefore transported along the production line in a movement direction extending longitudinally from upstream to downstream between and in the various stations dedicated to each process that the containers have to undergo.

In particular the containers must be fed to a processing machine via a container entry or a container feed in such a manner that the distance between two successive containers corresponds to the throughput of the machine, that is to say its processing speed. Also, there may be required a modification of the spacing interval of the successive containers during passage from one processing station to a following processing station.

Furthermore, it may be necessary to modify the orientation of the containers before entry into a processing machine, for example before entry into a filling machine or a labelling machine.

For orderly feeding and for spacing or separating the containers at a particular pitch, that is to say at the distance corresponding to the throughput of the machine, it is known to have recourse to a feed screw system adapted to pivot on itself and for example making it possible to increase the pitch between two containers fed successively.

It is also known to use such a screw for the separation of the containers, disposed alongside a transport path for the containers and therefore adapted to be driven in rotation. One of the problems with this type of feed screw is that it is liable to leave scratch marks on the body of the container transported in this way. In effect, a feed screw includes a thread, the crest of the thread coming into contact with the container to be fed, spaced and/or oriented, this contact then causing rubbing.

The disclosure advantageously proposes a feed screw unit and a feed screw in which the helical thread comprises at least two grooves, each of the at least two grooves being configured to receive a wear ring. The presence of the wear ring makes it possible to prevent direct contact between the container and the thread or with the body of the screw. The container being in contact with the wear ring, rubbing is reduced and the appearance or integrity of said container is not degraded.

The disclosure is directed first to a unit for a screw for feeding, setting the pitch of and/or setting the orientation of containers, said unit including:
a central body extending from an upstream end to a downstream end along a longitudinal axis,
a substantially helical thread rigidly attached to said body, said thread comprising a plurality of helixes, each helix having a crest, an upstream flank and a downstream flank, said thread being configured to be engaged between said containers.

The unit according to the disclosure is characterized in that it includes for each helix at least:
a first substantially helical groove at the level of the upstream flank of said helix adapted to receive a first wear ring, and
a second substantially helical groove at the level of the downstream flank of said helix adapted to receive a second wear ring.

Some embodiments of the feed screw unit include a third substantially helical groove at the level of the downstream flank of said helix adapted to receive a third wear ring.

In some embodiments the screw unit is produced in one piece.

In possible embodiments the screw unit is produced by 3D printing and is preferably made of polyamide.

In some embodiments the at least two grooves have the same helical profile as the helix.

In accordance with a possible additional feature in an axial plane perpendicular to the longitudinal axis of the body of the screw unit, the upstream angle of the upstream flank of the helix is identical to the downstream angle of the downstream flank of said helix.

The disclosure is also directed to a screw for feeding, setting the pitch of and/or setting the orientation of containers, said screw extending from an upstream end to a downstream end. The screw according to the disclosure is characterized in that it includes:
a central shaft,
at least one unit as described above, the shaft being inserted in the body of said unit.

In preferred embodiments the screw comprises at least two units, said units being abutted against one another by means of said central shaft inserted in the body of each of said at least two units so that the at least two grooves of each of said at least two units extend continuously from the upstream end to the downstream end.

In accordance with a possible additional feature the at least two grooves include one fixing point for a wear ring.

In some embodiments the screw includes at least two wear rings, said at least two wear rings having a shape complementary to the shape of the respective groove and extending the whole length thereof.

In accordance with a possible additional feature the at least two wear rings are made of polymer, preferably of polytetrafluoroethylene.

In some embodiments from the upstream end to the downstream end the helixes forming the thread have a decreasing radius and the pitch separating two successive helixes decreases.

The disclosure is further directed to an installation for processing containers including at least:
a device for conveying said containers,
an entry of a processing machine,
at least one rotary shaft.

The installation according to the disclosure is characterized in that it includes at least one feeding, pitch setting and/or orientation setting screw as described above, said screw being horizontal and extending from upstream to downstream in a direction along one side of said conveyor device, said at least one screw being fixed to said at least one rotary shaft in order to enable it to rotate.

In accordance with a possible additional feature the installation further includes a guide rail on the opposite side of and along said conveyor device, said rail facing said screw.

Another embodiment of the installation includes a second rotary shaft and a second screw, said second screw being on the opposite side of the conveyor device and extending in the upstream to downstream direction, said second screw being fixed to said second rotary shaft connected to said second screw in order to enable it to rotate, the two screws being symmetrical and configured to turn in opposite directions.

The disclosure also includes a method for feeding with containers an entry of a machine for processing said containers, said method including at least the following steps:
transporting the containers on a conveyor device,
setting the pitch of and/or setting the orientation of said containers before their entry into the processing machine, each body of a container being adapted to engage at the level of the upstream end a screw between two helixes formed on the perimeter of the body of said screw.

In the provided method, the step of setting the pitch and/or setting the orientation of the containers is implemented via at least one screw as described above.

Turning now to the drawings, exemplary embodiments are described in detail.

### EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

### Example 1

In the remainder of the description elements having an identical structure or analogous functions are designated by the same reference.

The disclosure is directed firstly to a unit 100 for a feed, spacing and/or orientation screw 1 configured to be used in an installation for packaging containers 2.

In the context of the disclosure the feed, spacing and/or orientation screw 1, referred to hereinafter as the feed screw 1, is intended to feed with containers 2 a processing machine 7. Some embodiments can also enable spacing or separation of the containers 2, which is to say that the screw 1 enables definition of a precise interval or distance that separates two successive containers 1, said containers 1 circulating on a conveyor device 6. This enables exact definition of distribution or feeding as a function of the requirements of the processing machine 7.

Finally, the screw 1 can modify the orientation of the circulating containers 2 before their entry into the processing machine 7.

The screw 1 acts in a similar manner to a cylindrical cam and could be referred to as a lead screw. Furthermore, the screw 1 is configured to be used in the conveyor device 6 on which the containers 2 circulate.

The containers 2 are offered up one after the other on the conveyor device 6 and enter continuously the thread 4 of the screw 1. When the containers 2 are taken up in the thread 4 some rubbing occurs, which can cause deterioration of the body 20 of said containers 2.

The containers 2 are by way of non-limiting example bottles, cans, small bottles, jerrycans or cartons. Such a container 2 is intended to contain a fluid, a liquid, powder or granules, in particular of agriculture-foodstuff or cosmetic type. This list is not exhaustive.

These containers 2 can be made of any type of material, metal, glass, but preferably a plastic-based material, notably polyethylene (PE) or polypropylene (PP), conferring a flexibility rendering said container 2 semi-rigid.

Such containers 2 can have any shape, symmetrical or not, regular or irregular. Furthermore, each of the containers 2 can have a rounded section, of circular or oval overall shape, or a polygonal section, notably a rectangular or square section.

In particular, the use of a container 2 of specific shape, for example asymmetrical or oblong, is widespread in the food, home care and personal care (FHPC) container field, for example for a container 2 intended to contain shower gel or shampoo, or in the field of home care and cleaning, such as a spray bottle for detergents and disinfectants.

In the context of the disclosure the unit 100 for a feed screw 1 includes:
a central body 3, or core, with an upstream end 300 and a downstream end 301 along a longitudinal axis X,
a substantially helical thread 4 rigidly attached to said body 3, said thread 4 comprising a plurality of helixes 40, each helix 40 having a crest 41, an upstream flank 42 and a downstream flank 43, said thread 4 being configured so that it can be engaged between said containers 2.

Furthermore, the axis of each helix 40 is parallel to the longitudinal axis X of the central body 3. The central body 3 is hollow, which is to say that it comprises an opening, cavity or orifice extending from the upstream end 300 to the downstream end 301.

In other words, the unit 100 comprises at least two helixes 40, or splines, with a helical profile.

The unit 100 is configured in such a manner that the containers 2 enter the thread 4 at the level of the hollow or space 44 separating two successive helixes 40 in order to be successively taken up by the unit 100.

The unit 100 is characterized in that it comprises for each helix 40 at least:
a first substantially helical groove 5a at the level of the upstream flank 42 of said helix 40, said first groove 5a being adapted to receive a first wear ring 50a,
a second substantially helical groove 5b at the level of the downstream flank 43 of said helix 40, said second groove being adapted to receive a second wear ring 50b.

The first groove 5a is on the upstream flank 42 of the helix 40, said upstream flank 42 being on the side of the upstream end 300 along the longitudinal axis X. In other words the upstream flank 42 includes at least one substantially helical groove 5a. The groove 5a is preferably continuous over one revolution of the unit 100 and shallow. For example the depth of the groove 5 a is about 6 millimeters (mm).

In a preferred embodiment the groove 5a is in the upper part of the upstream flank 42, which is to say that the groove 5a is near the crest 41.

The position of the at least one groove 5a is preferably determined as a function of the shape and the size of the containers 2 to be processed.

In some embodiments the number of grooves and the position thereof on the upstream flank 42 is determined as a function of the shape and size of the containers 2 to be processed.

The second groove 5b is on the downstream flank 43, said downstream flank 43 being on the side of the downstream end 301 along the longitudinal axis X. In other words the downstream flank 43 includes at least one substantially helical groove 5b.

The groove 5b is preferably continuous over one revolution of the unit 100 and shallow. For example, the depth of the groove 5 b is about 6 millimeters (mm).

In some embodiments the groove 5b is in the upper part of the downstream flank 43, which is to say that the groove 5b is near the crest 41.

In some embodiments the groove 5b is in the lower part of the downstream flank 43, which is to say that the groove 5b is near the hollow or space 44 between two helixes 40.

The position of the at least one groove 5b is preferably determined as a function of the shape and size of the containers 2 to be processed.

In some embodiments the number of grooves and the position thereof on the downstream flank 43 is determined as a function of the shape and size of the containers 2 to be processed.

In other words, each of the at least two grooves 5a, 5b is at a distance from the crest 41 of the helix 40 predefined as a function of the shape and dimensions of the container 2.

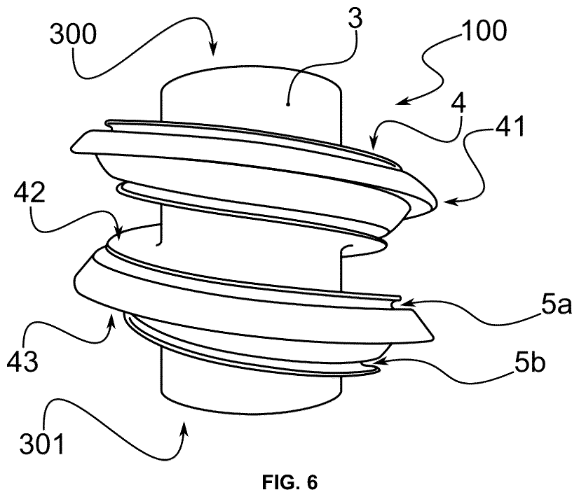

An embodiment of a unit 100 including two grooves 5a and 5b can be seen in FIG. 6. In particular the first groove 5a is near the crest 41 and the second groove 5b is in the lower part of the downstream flank 43. The two grooves 5a and 5b are respectively configured to receive a wear ring 5a and 5b. The presence of a wear ring enables prevention of direct rubbing between the container 2 and the body 3 or the helix 40 of the unit 100 and therefore enables prevention of scoring or galling. Furthermore, the wear ring 5a, 5b is removable and can easily be replaced if worn.

Furthermore, the dimensions of the corresponding groove 5a, 5b can vary as a function of the dimensions of the wear ring 5a, 5b to be inserted.

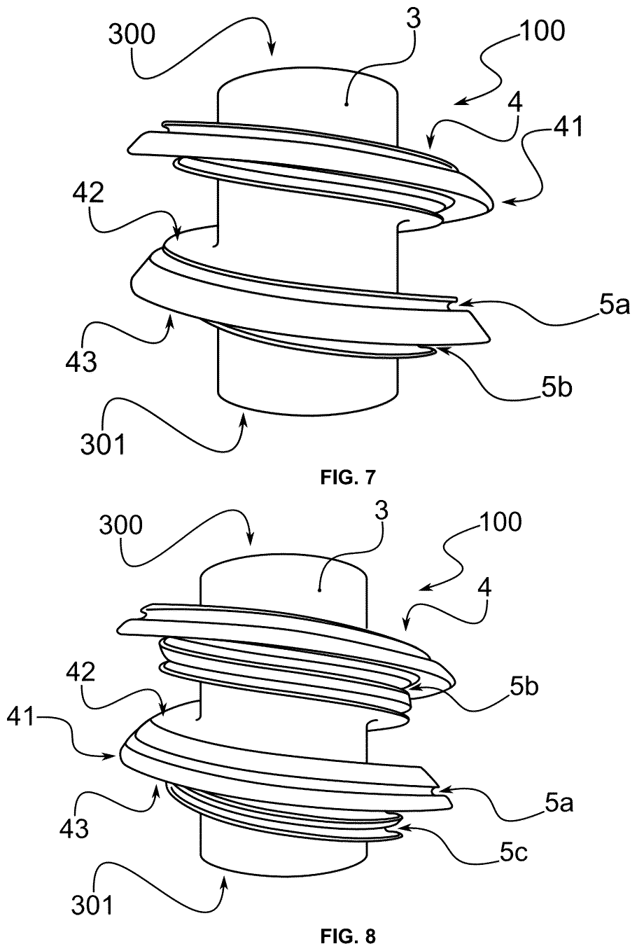

Another embodiment of a unit 100 including two grooves 5a and 5b can be seen in FIG. 7. In particular the first groove 5a is in the lower part of the upstream flank 42 and the second groove 5b is on the downstream flank 43 near the crest 41. The two grooves 5a and 5b are respectively configured to receive a wear ring 5a and 5b.

In these embodiments the unit 100 includes a third substantially helical groove 5c on the downstream flank 43, said groove 5c being adapted to receive a third wear ring 50c.

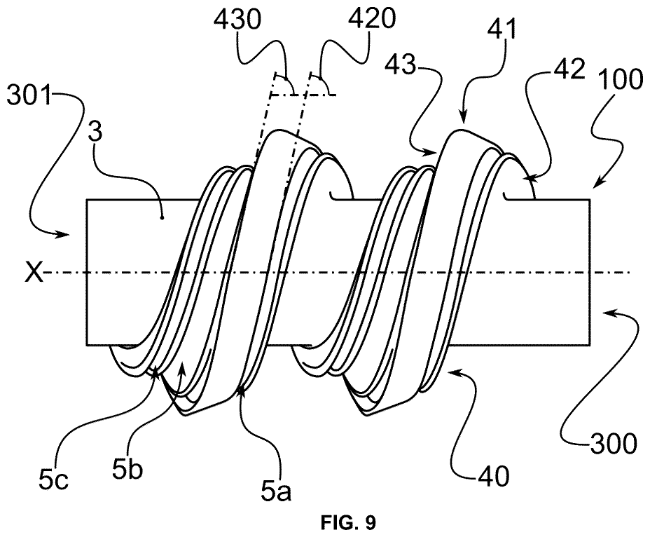

One embodiment can be seen in FIG. 8. This embodiment, in which the unit 100 includes three grooves 5a, 5b and 5c, is particularly suitable for containers of tall jerrycan type.

In some embodiments the unit 100 is made in one piece. For example, the unit 100 can be made by additive manufacture or 3D printing and in particular by melted filament deposition, multijet melting or selective laser sintering. The unit 100 is preferably made of polyamide, in particular of PA12 type nylon. This embodiment is particularly advantageous in that it enables great flexibility in sizing the unit 100 and in particular in sizing the helixes 40 and the at least two grooves 5a, 5b. It is therefore possible to produce screw units 100 suitable for any type of container 2.

In some preferred embodiments the at least two grooves 5a, 5b of the unit 100 have the same helical profile as the at least two helixes 40 of said unit 100. In other words, the at least two grooves 5a, 5b match the pitch p of the thread 4.

In accordance with one possible additional feature, in an axial plane the upstream angle 420 of the upstream flank 42 of each helix 40 is the same as the downstream angle 430 of the downstream flank 43 of said helix 40. The angle of the helix 40 is calculated in a plane perpendicular to the longitudinal axis X of the body 3 of the unit 100.

It is to be understood that the respective angles 420, 430 of the at least two helixes 40 of the unit 100 can be different.

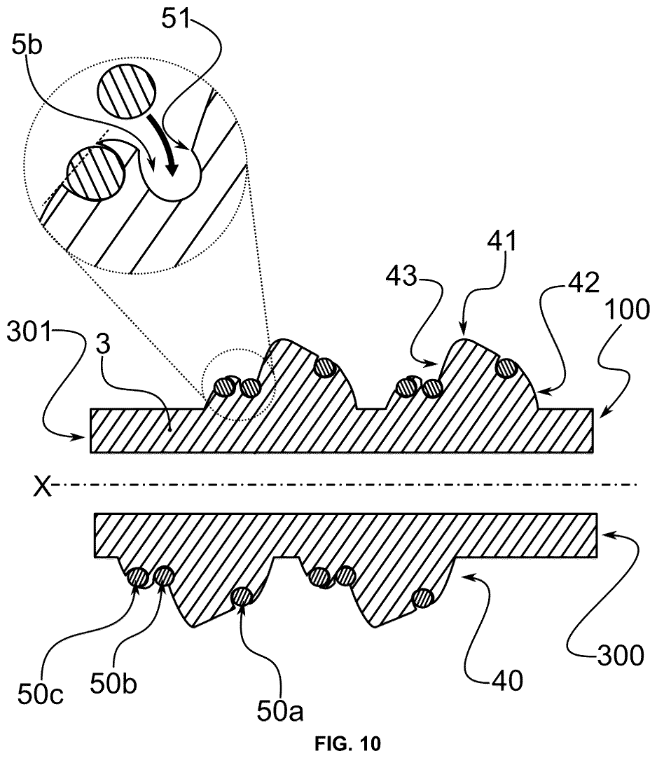

The helixes 40 of a unit 40 preferably have the same angles 420, 430. This embodiment can be seen in FIG. 9 in particular in which can be seen two successive helixes 40 with equivalent angles 420, 430 of inclination, the two angles 420 and 430 of the same helix 40 being substantially equal.

The inclination of the upstream flanks 42 and the downstream flanks 43 perpendicular to the longitudinal axis X of the body 3 is preferably determined as a function of the type of container 2 to be processed. For example, for a tall and narrow container 2 the inclination of the upstream flanks 42 and the downstream flanks 43 must be high, which is to say that the angles 420 and 430 must be acute angles. Conversely, for a low and wide container 2 the angles 420 and 430 must be obtuse angles. In other words the values of the angles of inclination 420 and 430 depend on the type of container 2 to be processed.

Furthermore, as described above the body 3 of a unit 100 is preferably hollow. Its dimensions are notably defined by its radius r1 calculated in a plane perpendicular to the longitudinal axis X.

In some embodiments the radius r1 of the body 3 is constant from the upstream end 300 to the downstream end 301.

In one possible variant the radius r1 of the body 3 increases or decreases from the upstream end 300 to the downstream end 301.

The disclosure also concerns a feed screw 1, said screw 1 extending from an upstream end 30 to a downstream end 31 along a longitudinal axis parallel to the longitudinal axis X. The screw 1 according to the disclosure is characterized in that:
it has a central shaft 101 extending from the upstream end 30 to the downstream end 31,
it includes at least one unit 100 as described above, said central shaft 101 being inserted in the opening of the body 3.

In one preferred embodiment the screw 1 comprises at least two units 100 as described above, said units 100 being abutted against one another by means of said shaft 101 inserted in the body 3 of each of said at least two units 100 so that the at least two grooves 5a, 5b of each of said at least two units 100 extend in a continuous manner from the upstream end 30 to the downstream end 31 along the central shaft 101.

In the context of the disclosure the feed screw 1 is intended for a device 6 for conveying containers 2.

The screw 1 is therefore preferably produced by a succession of units 100 abutted against one another, each unit 100 being identical. The screw units 100 therefore constitute screw modules, the screw 1 being modular. In effect, given the speed at which the containers 2 are made, their size and also the throughput of the processing machine, the screw 1 can be very long. It can therefore be necessary to abut units 100 together to constitute a feed screw 1.

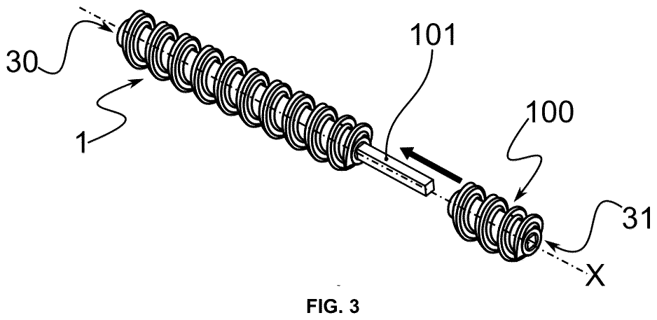

There can be seen in FIG. 3 an example of the production of such a screw 1 with a plurality of units 100 abutted against one another. In other words, the downstream end 301 of the upstream unit 100 is adjacent to the upstream end 300 of the adjacent downstream unit 100 along the central shaft 101.

In the embodiment represented in FIG. 3 the units 100 have the same characteristics, which is to say that the units 100 include threads 4 having the same helical profile. In other words, each helix 40 of the screw 1 has the same radius r2 and the thread 4 has the same pitch p between adjacent helixes 40 of said screw 1 in the direction X.

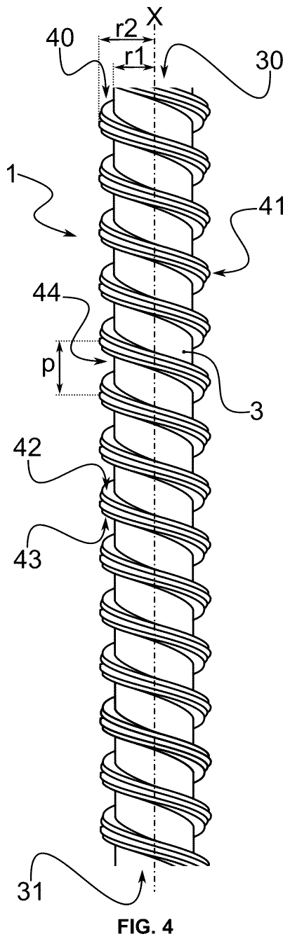

In FIG. 4, which depicts this embodiment, it can be seen that each helix 40 of the thread 4 has the same radius r2, which is to say that the crest 41 is situated at the same distance relative to the central shaft 101 for each helix 40. The pitch p is therefore constant between the helixes 40 from the upstream end 30 to the downstream end 31 of the screw 1 along the longitudinal axis X. This embodiment is particularly suitable for containers 2 of cylindrical bottle type.

It can be seen in FIG. 3 that the units 100 are abutted so that the helixes 40 exhibit continuity. In other words, an upstream unit 100 has its downstream end 301 against the upstream end 300 of the adjacent downstream unit 100, and vice versa. In a manner that is not represented the units 100 are abutted so that the grooves 5a, 5b form a continuity from the upstream end 30 to the downstream end 31 along the central shaft 101.

It is to be understood that the central shaft 101 of the screw 1 is preferably inserted in the body 3 of at least two units 101 so that they are abutted. The shaft 101 is therefore parallel to the longitudinal axis X of a unit 100 for the screw 1.

The units 100 abutted against one another to form a screw 1 preferably have different characteristics, which is to say that the helixes 40 of at least two units 100 of the screw 1 have:
a different radius r2 and/or
the thread 4 has a pitch p varying along the screw 1, along and relative to the central shaft 101.

By radius r2 is meant the distance between the crest 41 of the helix and the central shaft 101 of the screw 1.

By pitch p is meant the distance between two successive crests 41.

Furthermore, in some embodiments the radius r1 of the body 3 of the unit 100 is constant from the upstream end 30 to the downstream end 31 of the screw 1.

In one possible variant the radius r1 of the body 3 of the unit 100 increases or decreases from the upstream end 30 to the downstream end 31 of the screw 1.

It is clear that when the screw 1 comprises at least two units 100 and said units 100 can be abutted in such a manner that the radius r1 is constant or increases or decreases from the upstream end 30 to the downstream end 31 of the screw 1.

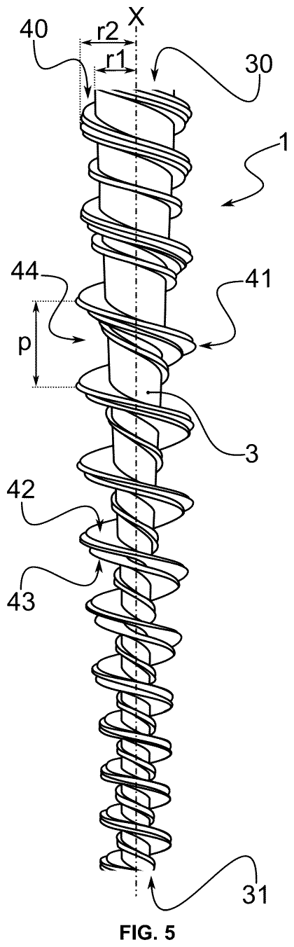

An example of this embodiment is depicted in FIG. 5, in which a screw 1 comprising a plurality of units 100, said units 100 being abutted so that along the central shaft 101 from the upstream end 30 to the downstream end 31:
the helixes 40 of the units 100 have decreasing radii r2, and
the bodies 3 of the units 100 have decreasing radii r1.

Furthermore, in this illustrative example the pitch p separating two successive helixes 40 also decreases.

This embodiment is very particularly suitable for orienting jerrycan type containers 2, the first helixes 40 having a larger pitch p enabling the jerrycan to be taken up in the space 44 and the radius r2 enabling orientation of the jerrycan, the screw 1 rotating clockwise or anticlockwise causing it to pivot so that in moving forward the jerrycan is turned 90° and held between two successive helixes 40 in a narrower space 44.

In other words in some embodiments the pitch p between the crests 41 of two successive helixes 40 or the space 44 between two helixes 40, that is to say the space 44 between the base of the downstream flank of the upstream helix 40 and the base of the upstream flank of the downstream helix 40, decreases between the upstream end 30 and the downstream end 31 of the screw 1.

For example, for a jerrycan type container 2 having a length of about 144 millimeters (mm), a width of about 67 millimeters (mm) and a height of about 281 millimeters (mm) the radius r2 is between about 50 millimeters (mm) and about 62 millimeters (mm) and the upstream angle 420 of the upstream flank 42 and the downstream angle 430 of the downstream flank 43 decreases between the upstream end 30 and the downstream end 31, between about 40 degrees upstream to about 15 degrees downstream. This decrease of the upstream angle 420 and the downstream angle 430 enable a change of the orientation of the jerrycan upstream of being taken up followed by assuming the correct pitch downstream.

In alternative embodiments the pitch p between the crests 41 of two successive helixes 40 or the space 44 between two helixes 40 increases between the upstream end 30 and the downstream end 31 of the screw 1.

In some embodiments in at least one unit 100 of the screw 1 the at least two grooves 5a, 5b include at least one point 51 of inflexion called the fixing point 51 for fixing each of the wear rings 50a, 50b. The first unit 100 at the upstream end 30 and the last unit 100 at the downstream end 31 preferably each include a point 51 of inflexion for the wear rings 50a, 50b. Thus, when inserting the wear ring 50a, 50b in the respective groove 50a, 50b the wear ring undergoes plastic deformation at the level of the point 51 of inflexion. Once inserted the wear ring 50a, 50b is firmly retained in the corresponding groove 5a, 5b.

In embodiments in which the unit 100 includes three grooves 5a, 5b, 5c for at least one unit 100 of the screw 1 each of the grooves 5a, 5b, 5c includes at least one point 51 of inflexion for the wear rings 50a, 50b, 50c.

FIG. 10 represents an illustrative example of a unit 100 for a screw 1 according to the disclosure in which the helixes 40 includes three substantially helical grooves 5a, 5b, 5c into which are respectively fixed the wear rings 50a, 50b, 50c. The point 51 of inflexion is also represented. It can be seen that the wear rings 50a, 50b, 50c project.

In effect a peripheral part of the wear ring 50a, 50b, 50c preferably projects in such a manner as to come into contact with a peripheral wall of the container 2 to be transported and/or to orient it. The presence of the wear rings 50a, 50b, 50c prevents direct rubbing between the feed screw 1 and the body 30 of the containers 2.

In some embodiments the screw 1 includes at least two wear rings 50a, 50b, said at least two wear rings 50a, 50b having a shape complementary to the shape of the respective groove 5a, 5b and extending the entire length thereof, that is to say from the upstream end 30 to the downstream end 31 along the central shaft 101 or along the longitudinal axis X.

This embodiment is particularly advantageous because it enables protection of the containers 2 during manipulation thereof by the screw 1. Also, the wear rings 50a, 50b are easily replaceable, in particular in the event of wear.

In some embodiments the screw 1 includes three wear rings 50a, 50b and 50c. The three wear rings 50a, 50b and 50c preferably have a shape complementary to the shape of the respective groove 5a, 5b, 5c and extend the whole length thereof, that is to say from the upstream end 30 to the downstream end 31 along the central shaft 101 or along the longitudinal axis X.

Depending on the type of container 2, that is to say depending on their format, type or material, the screw 1 may include two or three or even more wear rings.

In preferred embodiments the at least two wear rings 50a, 50b are made of polymer, preferably of polytetrafluoroethylene.

The disclosure is also directed to a packaging installation 10 including at least:
a device 6 for conveying containers 2,
an entry 70 of a processing machine 7,
at least one rotary shaft 8.

The installation 10 is characterized in that it includes at least one feed, pitch setting and/or orientation setting screw 1 as described above. The screw 1 according to the disclosure is horizontal and extends from upstream to downstream in the direction D. In other words, the central shaft 101 of the screw 1 or the longitudinal axis X is parallel to the conveying direction D. The screw 1 is situated along the conveyor device 6 and is fixed onto a rotary shaft 8 in order to enable it to be rotated. In particular the central shaft 101 inserted in the screw 1 cooperates with the shaft 8.

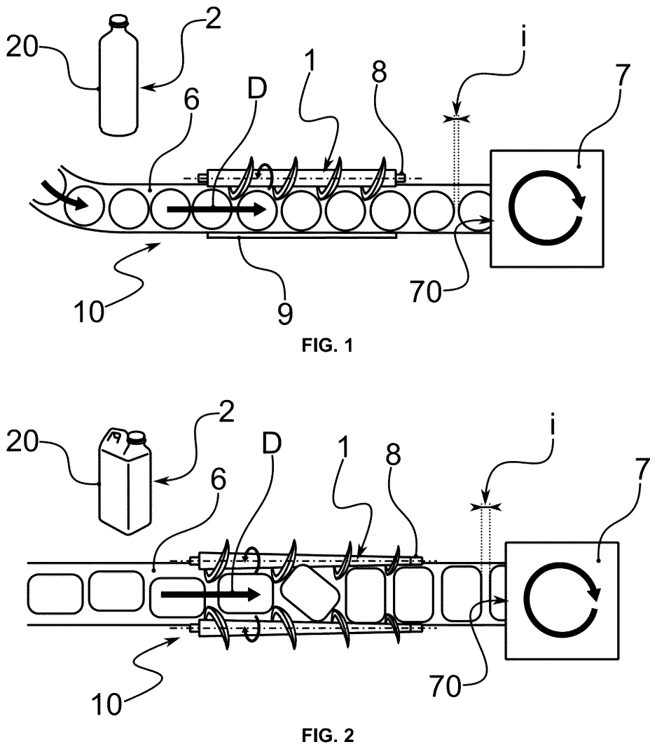

An illustrative example of such an installation 10 can be seen in FIG. 1.

The conveyor device 6 includes driving means such as a so-called endless conveyor belt on which the containers 2 rest. The containers 2 circulate in single file, that is to say one behind the other, in a vertical position on their bottom, and may or may not all have the same orientation. Furthermore, two successive containers 2 are separated by an interval i that corresponds to the distance that separates them.

The containers 2 circulate on this conveyor device 6, for example from a preceding station or from an accumulation table to the entry 70 of the processing machine 7. The processing machine 7 can be any type of machine 7 present on a packaging line, for example a labelling machine, a filling machine or a film-wrapping tunnel.

The entry 70 of the machine 7 may include a transfer star, not represented, said star being downstream of the feed screw 1 and rotating synchronously with it.

In some embodiments the installation 10 includes along the conveyor device 6 a lateral guide rail 9 situated on the opposite side and facing the feed screw 1, as represented in FIG. 1. The body 20 of the container 2 slides against the guide rail 9 on one side and is engaged with the screw 1 on the other side of the conveyor device 6.

Thus containers 2 circulate on the conveyor device 6 and are guided by the guide rail 9, which is for example of a sliding type, while being taken up by the screw 1, penetrating at least partially into the space 44 separating two successive helixes 40. The substantially helical profile of the screw 1 favors the taking up of the containers 2 between the helixes 40 of the thread 4. The containers 2 held in this way can advantageously be oriented, spaced or moved closer together as a function of the requirements of the processing machine 7.

In some embodiments the installation 10 includes a second screw 1 and a second rotary shaft 8. As depicted diagrammatically in FIG. 2 the second screw 1 is on the opposite side of the conveyor device 6 and extends from upstream to downstream in the conveying direction D. The second screw 1 is fixed to the second rotary shaft 8 connected to the body 3 of said second screw 1 in order to enable it to be rotated. In particular the two screws 1 are symmetrical and are configured to turn in opposite directions.

Finally the disclosure is directed to a method for feeding containers 2 to an entry 70 of a machine 7 for processing said containers 2, the method including at least the following steps:
transporting the containers 2 on a conveyor device 6,
setting the pitch and/or orientation of the containers 2 before they enter the processing machine 7, each body 20 of a container 2 being adapted to be engaged at the level of the upstream end 31 of a screw 1 between two helixes 40 formed on the perimeter of the body 3 of said screw.

In other words, the containers 2 are adapted to be taken up between two successive helixes 40 along the screw 1.

The method according to the disclosure is characterized in that the step of setting the pitch and/or the orientation of the containers 2 is implemented by means of at least one feed screw 1 as described above.

The containers 2 are therefore transported on the conveyor device 6 and spaced upstream by a given interval i. They are then taken up by at least one feed screw 1 that rotates on itself. Each container 2 penetrates at least partially into a space 44 between two successive helixes 40. The screw 1 rotating, the container 1 can be accelerated or decelerated as a function of the speed difference between the rotation speed of the screw 1 and the conveyor device 6. This speed difference has the effect of either increasing the interval i between two successive containers 2 or reducing that interval i. The presence of the at least two grooves 5a, 5b on said screw 1 enables insertion therein of at least two wear rings 50a, 50b, a wear ring 50a, 50b being placed in each respective groove 5a, 5b. These at least two wear rings 50a, 50b protect the body 20 of the containers 2 during manipulation of said containers 2 and are easily replaceable in the event of wear.

The screw 1 is also able to change the orientation of a container 2. When the container 2 is taken up by the screw 1 at the level of the upstream end 30 in a given orientation it is therefore discharged at the level of the downstream end 31 in an orientation differing from that at the outset.

The method according to the disclosure is therefore adapted to be implemented in a packaging installation 10 as described above.

Reciprocally, the installation 10 as described above is able to implement the method according to the disclosure.

The disclosure advantageously enables provision of a feed screw 1 able to feed a machine 7 for processing containers 2 effectively and without risk of spoiling the body 20 of said containers 2.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. In an embodiment, “about 0 ” can refer to 0, 0.001, 0.01, or 0.1. In an embodiment, the term “about” can include traditional rounding according to significant figures of the numerical value. In addition, the phrase “about ‘x’ to ‘y’” includes “about ‘x’ to about ‘y’”.

Therefore, at least the following is claimed:

1. A unit for a screw for feeding, setting a pitch of and/or setting an orientation of containers, the unit comprising:
a central body extending from an upstream end to a downstream end along a longitudinal axis;
a substantially helical thread rigidly attached to the body, the thread comprising a plurality of helixes, each helix having a crest, an upstream flank and a downstream flank, the thread being configured to be engaged between the containers; and
for each helix at least:
a first substantially helical groove at a level of the upstream flank of the helix configured to receive a first wear ring, and
a second substantially helical groove at a level of the downstream flank of the helix configured to receive a second wear ring.

2. The unit according to claim 1, further comprising a third substantially helical groove at the level of the downstream flank of the helix configured to receive a third wear ring.

3. The unit according to claim 1, wherein the unit is produced in one piece.

4. The unit according to claim 1, wherein the unit is produced by 3D printing and is made of polyamide.

5. The unit according to claim 1, wherein the first substantially helical groove and the second substantially helical groove have a same helical profile as the helix.

6. The unit according to claim 1, wherein, in an axial plane perpendicular to a longitudinal axis of the body, an upstream angle of the upstream flank of the helix is identical to a downstream angle of the downstream flank of the helix.

7. A screw for feeding, setting a pitch of and/or setting an orientation of containers, the screw extending from an upstream end to a downstream end, the screw comprising:
a central shaft; and
at least one unit, the unit comprising:
a central body extending from an upstream end to a downstream end along a longitudinal axis;
a substantially helical thread rigidly attached to the body, the thread comprising a plurality of helixes, each helix having a crest, an upstream flank and a downstream flank, the thread being configured to be engaged between the containers; and
wherein each helix comprises at least two grooves, the two grooves comprising:
a first substantially helical groove at a level of the upstream flank of the helix configured to receive a first wear ring, and
a second substantially helical groove at a level of the downstream flank of the helix configured to receive a second wear ring,
wherein the shaft is inserted in the body of the unit.

8. The screw according to claim 7, comprising at least two units, the units being abutted against one another via the central shaft inserted in the body of each of the at least two units so that the at least two grooves of each of the at least two units extend continuously from the upstream end to the downstream end.

9. The screw according to claim 7, wherein the at least two grooves include one fixing point for a wear ring.

10. The screw according to claim 7, comprising at least two wear rings, the at least two wear rings having a shape complementary to a shape of the respective groove and extending a whole length thereof.

11. The screw according to claim 10, wherein the at least two wear rings are made of polytetrafluoroethylene.

12. The screw according to claim 7, wherein, from the upstream end to the downstream end:
The plurality of helixes forming the thread have a decreasing radius, and
a pitch separating two successive helixes decreases.

13. A conveyor installation for processing containers comprising at least:
a conveyor device for conveying the containers;
an entry of a processing machine; and
at least one rotary shaft,
wherein the installation comprises at least one screw according to claim 7, the screw being horizontal and extending from upstream to downstream in a direction along one side of the conveyor device, the at least one screw being fixed to the at least one rotary shaft in order to enable the screw to rotate.

14. The conveyor installation according to claim 13, wherein the screw is selected from one or more of a feeding screw, a pitch setting screw, and an orientation setting screw.

15. The conveyor installation according to claim 13, further comprising a guide rail on an opposite side of and along the conveyor device, wherein the guide rail faces the screw.

16. The conveyor installation according to claim 13, further comprising a second rotary shaft and a second screw, the second screw being on the opposite side of the conveyor device and extending in the upstream to downstream direction, the second screw being fixed to the second rotary shaft connected to the second screw in order to enable the second rotary shaft to rotate, the first and second screws being symmetrical and configured to turn in opposite directions.
